(19) United States
(12) Patent Application Publication
(10) Pub. No.: US 2022/0283324 A1
(43) Pub. Date: Sep. 8, 2022

(54) RADIATION IMAGING APPARATUS AND RADIATION IMAGING SYSTEM

(71) Applicant: CANON KABUSHIKI KAISHA, Tokyo (JP)

(72) Inventor: Akehiko Uchiyama, Kanagawa (JP)

(21) Appl. No.: 17/652,539

(22) Filed: Feb. 25, 2022

(30) Foreign Application Priority Data
Mar. 4, 2021 (JP) 2021-034665

(51) Int. Cl. G01T 1/20; H04N 5/365

(52) U.S. Cl. CPC G01T 1/2006; G01T 1/20183; H04N 5/3651

(57) ABSTRACT

A radiation imaging apparatus comprising: a scintillator; a plurality of pixels configured to respectively detect light converted by the scintillator from radiation; and a corrector configured to perform a correction process on signal data based on signals output from the plurality of pixels is provided. The corrector is configured to perform a first correction process for acquiring a gain map for gain correction without placing an object, and a second correction process including gain correction using the gain map. Correction processes performed on dotted noise that occur at random both temporally and spatially are different for the first correction process and the second correction process.

### BACKGROUND OF THE INVENTION

### Field of the Invention

The present invention relates to a radiation imaging apparatus and a radiation imaging system.

### Description of the Related Art

There are widely used radiation imaging apparatuses including a flat type sensor panel having pixels arranged thereon in an array, the pixels being formed of a combination of photoelectric conversion elements and switching elements. Japanese Patent Laid-Open No. 2002-026302 describes using a fluorescent plate to convert incident radiation into light that can be detected by an imaging element formed on a semiconductor substrate.

### SUMMARY OF THE INVENTION

There is a case where radiation transmits through a scintillator (fluorescent plate) without being converted into light that can be detected by an imaging element and, upon being incident on the imaging element, generates an electron-hole pair in the imaging element that received the incident radiation. Generation of the electron-hole pair may cause a bright spot pixel having an abnormally higher signal value than other pixels in the acquired image data. Such bright spot pixels appear at random as dots, both temporally and spatially.

Bright spot pixels, which occur at random while capturing and observing a video image, such as in fluoroscopic imaging, are annoying but may turn out to be negligible when observing an object. On the other hand, respective pixels on a sensor panel in a radiation imaging apparatus exhibit variation in operating characteristics for each pixel, and therefore a gain map is generated based on the image data acquired by irradiating radiation without placing an object, and a gain correction process is performed thereafter. In a case where bright spot pixels have mixed in the image data when generating the gain map, the bright spot pixels may affect the image data subjected to the gain correction process using the gain map having bright spot pixels mixed therein. Although it is necessary to reduce the effect of bright spot pixels in generation of the gain map, the demand for capturing and displaying an image (gain map generation) in real time may decrease in comparison with imaging for observing the object.

Some of the embodiments of the present invention provide an advantageous technique for reducing the effect of bright spot pixels in a radiation imaging apparatus.

According to some embodiments, a radiation imaging apparatus comprising: a scintillator; a plurality of pixels configured to respectively detect light converted by the scintillator from radiation; and a corrector configured to perform a correction process on signal data based on signals output from the plurality of pixels, wherein the corrector is configured to perform a first correction process for acquiring a gain map for gain correction without placing an object, and a second correction process including gain correction using the gain map, and correction processes performed on dotted noise that occur at random both temporally and spatially are different for the first correction process and the second correction process, is provided.

Further features of the present invention will become apparent from the following description of exemplary embodiments (with reference to the attached drawings).

### BRIEF DESCRIPTION OF THE DRAWINGS

FIG. 1 is a block diagram illustrating a configuration example of a radiation imaging apparatus according to the present embodiment;

FIG. 2 illustrates signal data acquired by the radiation imaging apparatus of FIG. 1;

FIG. 3 is an explanatory diagram of the flow of data processing performed by the radiation imaging apparatus of FIG. 1;

FIG. 4 illustrates signal data acquired by the radiation imaging apparatus of FIG. 1;

FIG. 5 is an explanatory diagram of transition of signal values of the signal data acquired by the radiation imaging apparatus of FIG. 1;

FIG. 6 is an explanatory diagram of processing of temporal filtering performed by the radiation imaging apparatus of FIG. 1;

FIG. 7 is an explanatory diagram of a flow of data processing performed by the radiation imaging apparatus of FIG. 1; and

FIG. 8 is an explanatory diagram of processing of temporal filtering performed by the radiation imaging apparatus of FIG. 1.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

In addition, radiation mentioned in the present invention may include α-rays, β-rays, γ-rays or the like, which are beams formed of particles (including photons) emitted by radioactive decay, and also include beams having a same or higher degree of energy with those beams such as, for example, X-rays, particle beams, or cosmic rays.

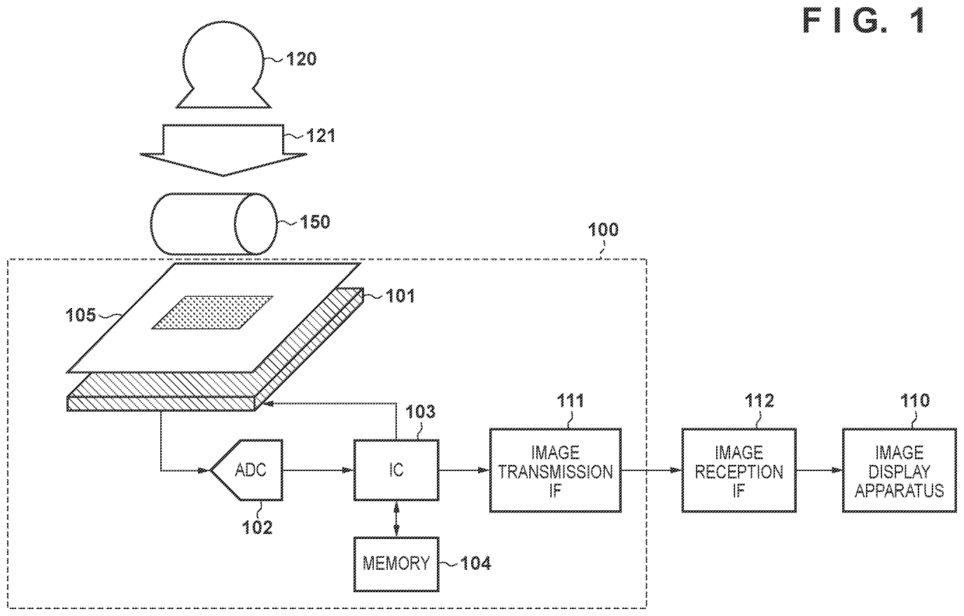

Referring to FIGS. 1 to 8, there will be described a configuration of a radiation imaging apparatus of the present embodiment, and a correction process performed thereby. FIG. 1 is a block diagram illustrating a configuration example of a radiation imaging system SYS according to the present embodiment. The radiation imaging system SYS includes a radiation imaging apparatus 100, an image display apparatus 110 configured to display image data acquired by the radiation imaging apparatus 100, and a radiation source 120 configured to irradiate radiation 121 on the radiation imaging apparatus 100. The radiation imaging apparatus 100 and the image display apparatus 110 are connected using wired or wireless communication. In addition, the radiation imaging apparatus 100 and the radiation source 120 may be connected using wired or wireless communication. The radiation imaging system SYS is intended to display a radiation image on the image display apparatus 110 in accordance with the radiation amount of the radiation 121 incident on the radiation imaging apparatus 100 from the radiation source 120 via an object 150.

The radiation imaging apparatus 100 may include a scintillator 105, a sensor array 101 having a plurality of pixels arranged thereon that respectively detect light converted by the scintillator 105 from radiation, and an integrated circuit (IC) 103 configured to control operation or the like of the sensor array 101. Furthermore, the radiation imaging apparatus 100 may include a memory 104. The IC 103, which will be described in detail below, includes a corrector configured to perform a correction process on signal data based on signals output from a plurality of pixels arranged on the sensor array 101.

When capturing a radiation image, an arrangement of the object 150 and the radiation imaging apparatus 100 is first determined so that the radiation 121 irradiated from the radiation source 120 is transmitted through the object 150 and projected on the radiation imaging apparatus 100. The radiation 121, a part of which being absorbed and shielded by the object 150, is incident on the scintillator 105 of the radiation imaging apparatus 100. The scintillator 105 emits light with a luminance corresponding to the radiation amount of the incident radiation 121. Although the sensor array 101 is illustrated in FIG. 1 in a manner apart from the scintillator 105, it may be stacked in close contact with the scintillator 105. Distribution of light emission from the scintillator 105 is detected by a plurality of pixels arranged on the sensor array 101.

The sensor array 101 has a plurality of pixels arranged thereon, as described above. Each of the plurality of pixels may include, for example, a photoelectric conversion element that detects light converted by the scintillator 105 from radiation, and a scanning switch for reading signals generated by the photoelectric conversion element. A semiconductor substrate, for example, may be used for the sensor array 101, the substrate having formed thereon a photodiode as the photoelectric conversion element, and a transistor as the scanning switch. In addition, a substrate made of glass or plastic, for example, may be used for the sensor array 101, the substrate having formed thereon a semiconductor layer. The semiconductor layer may have formed thereon a photodiode as the photoelectric conversion element, and a thin film transistor (TFT) as the scanning switch.

Signals for controlling operation of the sensor array 101, such as signals indicating switching of the scanning switch, may be generated by the IC 103 provided within the radiation imaging apparatus 100. With respect to the IC 103, a Field-Programmable Gate Array (FPGA), an Application Specific Integrated Circuit (ASIC) or the like may be used therefor. Analog signals output from the sensor array 101 are converted into digital data by an AD converter (ADC) 102 and input to the IC 103. As such, the IC 103 causes the pixels arranged on the sensor array 101 to generate signals, and performs control of signal-reading scan and reception of digital data in parallel. Therefore, it is possible to recognize which position on the sensor array 101 the pixel corresponding to the received digital data is located, and generate image data for displaying a radiation image by aligning the digital data accordingly. The IC 103 may store the image data in a memory 104, or output it to the outside of the radiation imaging apparatus 100 via an image transmission IF 111. The image data output from the image transmission IF 111 is transferred to the image display apparatus 110 via an image reception IF 112, and displayed as a radiation image based on the image data.

For example, video image capturing can be performed by repeating, with a desired period, scanning by the sensor array 101, transfer of image data, and display of radiation image. When capturing a video image, a case is conceivable in which an operator may instruct the object to move, in accordance with the radiation image visually recognized by the operator. In such a case, real time performance is required, and digital data generated by the ADC 102 must be transferred to the image display apparatus 110 in a shorter time.

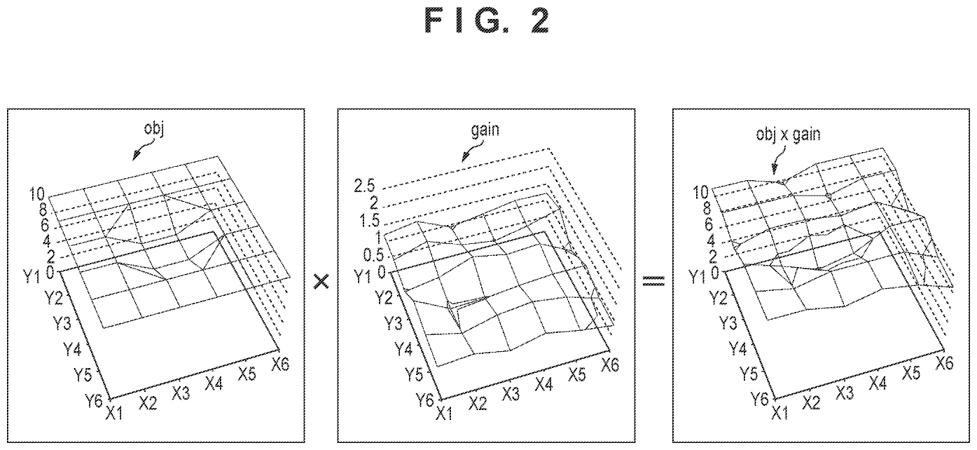

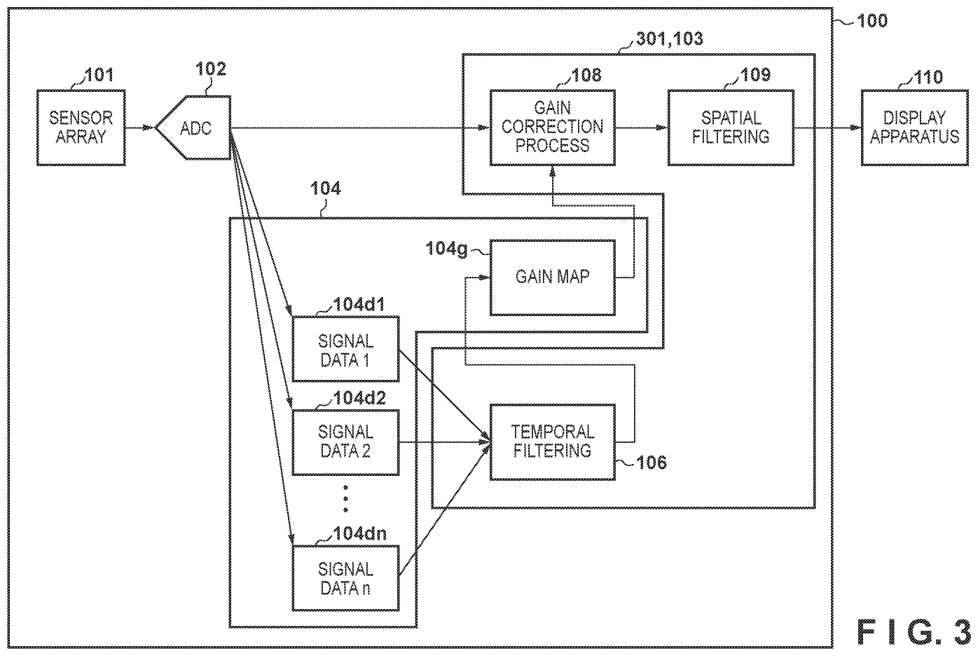

FIG. 2 is an explanatory diagram of signal data output from the ADC 203 based on signals output from a plurality of pixels. An object image obj in FIG. 2 represents a distribution of the radiation amount of radiation 121 incident on the scintillator 105. The object image obj in FIG. 2 has an object 150 placed at the center thereof, which indicates a smaller radiation amount incident on the center of the sensor array 101. However, the signal data output from the ADC 203 does not necessarily indicate the shape of the object image obj. This is because the sensitivity characteristics of each of the plurality of pixels arranged on the sensor array 101 are not uniform. A sensitivity distribution gain in FIG. 2 indicates an example of sensitivity distribution of all the entire pixels on the sensor array 101. The signal data output from the ADC 203 turn out to be data acquired by multiplicationally modulating the object image obj by the sensitivity distribution gain of the pixels arranged on the sensor array 101. As a result of the object image obj being modulated by the sensitivity distribution gain, data obj×gain illustrated in FIG. 3 is acquired as signal data. Accordingly, there is a possibility that distribution of the sensitivity characteristics gain remains in the data obj×gain output from the ADC 102, whereby it may become difficult to distinguish the original object image obj.

Restoration of the object image obj from the data obj×gain requires reverse conversion, i.e., division of the modulation process illustrated in FIG. 2. A mechanism for realizing restoration of the object image obj will be described, referring to FIG. 3. FIG. 3 is an explanatory diagram of the flow of the data processing performed on the signal data by the IC 103 of the radiation imaging apparatus 100.

As illustrated in FIG. 3, the IC 103 has provided thereon a corrector 301 configured to perform a correction process on signal data based on signals output from a plurality of pixels of the sensor array 101. The corrector 301 performs a correction process of restoring the object image obj from the data obj×gain. The sensitivity distribution gain is preliminarily stored in the memory 104 as a gain map 104g before capturing an image of the object 150.

When capturing an image of the object 150, the signal data (e. g., data obj×gain) subjected to analog-digital conversion by the ADC 102 is transferred to the corrector 301 of the IC 103, where a gain correction process 108 is performed. In the gain correction process 108, the gain map 104g is read from the memory 104 in response to transferring the data obj×gain from the ADC 102 to the corrector 301. Upon the data obj×gain and the gain map 104g corresponding to respective pixels of the sensor array 101 being transferred to the corrector 301, the corrector 301 divides the data obj×aain using the gain map 104g. Accordingly, it is possible to acquire the object image obj, for which modulation by a non-uniform sensitivity characteristics gain has been compensated, as image data. As has been described above, it is required in video image capturing or the like to reduce the time from when signal data is output from the ADC 102 to when it is transferred as image data to the image display apparatus 110. Therefore, the reading speed of the gain map 104g needs to be approximately the same as the speed of reading signal data from the ADC 102, in order to maintain the system operation speed of the radiation imaging system SYS (radiation imaging apparatus 100).

Next, there will be described a method (gain calibration) for acquiring the gain map 104g for gain correction. As has been described above, the signal data acquired from the ADC 102 turns out to be a multiplication of the distribution of the amount of radiation incident on the scintillator 105 (object image obj) and the sensitivity distribution gain of the pixels of the sensor array 101. Therefore, it turns out that, in principle, the signal data, which has been acquired in a state in which the distribution of the amount of radiation incident on the scintillator 105 has been maintained to be uniform without placing an object 150, may include as components thereof only the sensitivity distribution of the pixels of the sensor array 101. Normalizing the signal data acquired in this manner allows for acquisition of the gain map 104g.

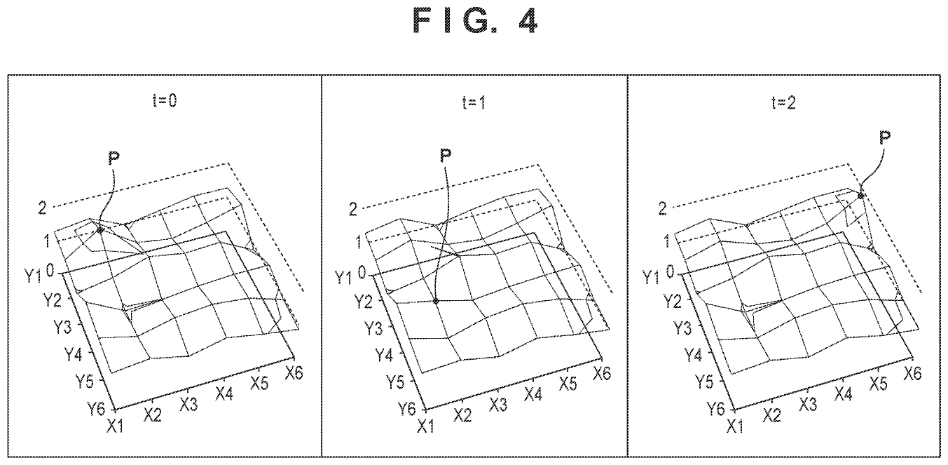

However, the signal data acquired while actually irradiating the radiation uniformly may be mixed with elements other than the sensitivity distribution of the sensor array 101. FIG. 4 illustrates a result of uniformly irradiating the radiation and acquiring signal data for multiple times (multiple frames). In FIG. 4, signal data acquired at three time points t=0, 1 and 2 are illustrated. Hereinafter, these signal data may be referred to as gain map original images. In the three pieces of signal data illustrated in FIG. 4, the signal values of the locations indicated by a black circle P are observed to be higher than the original sensitivity distribution gain illustrated in FIG. 2. This is a result of the radiation transmitted through the scintillator 105 being directly incident on a semiconductor layer such as the photoelectric conversion element of the sensor array 101 without being converted into light by the scintillator 105, thereby generating electron-hole pairs, and thus generating bright spots. When a gain map is generated from data including such bright spots, which are dotted noise, it turns out that excessive division is always performed on pixels at specific positions in a gain correction process using a gain map including bright spots, resulting in occurrence of dark spots in the corrected image. The dark spots keep occurring at specific fixed positions in the radiation image, and turn out to be a significant obstacle for human vision characteristics. In other words, image quality of radiation images acquired by the radiation imaging apparatus 100 may significantly decrease.

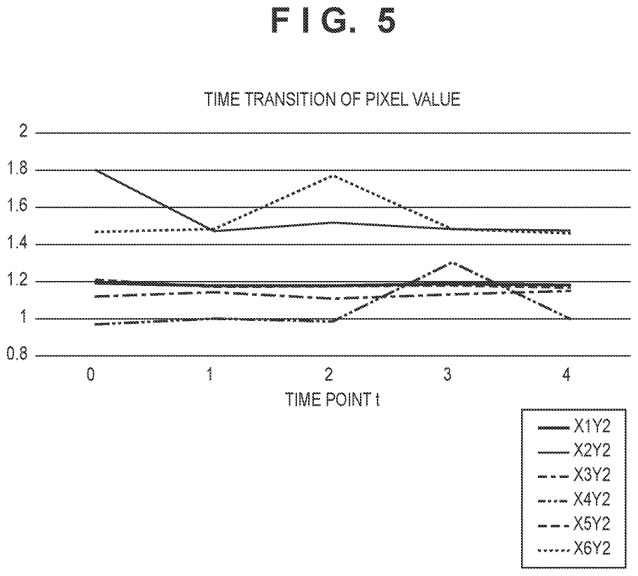

Therefore, let us consider a method for effectively removing bright spots, which are dotted noise, from the gain map. Bright spots, as illustrated in FIG. 4, occur at random both temporally and spatially, and therefore tend to occur at different positions each time the sensor array 101 is scanned. In FIG. 4, this is indicated by change of the position of the black circle P in accordance with the time point t. The same phenomenon as viewed from another angle will be described, referring to FIG. 5. FIG. 5 is a plot of the output of signal values of signal data of pixels arranged at positions XnY2 (n=1 to 6) of the sensor array 101 along with change of the time point t. The plots illustrated in FIG. 5 provide several findings. First, for pixels with no bright spots appearing, there is little temporal variation of the signal value of the signal data and therefore the signal value is stable (e.g., position X1Y2). In addition, even for pixels with bright spots appearing, signal values of the signal data with bright spots excluded therefrom have as little variation as pixels with no bright spots appearing. In addition, a signal value of a bright spot does not necessarily exceed the signal values of other pixels (e.g., signal value of position X4Y2 at time point t=3<signal value of position X2Y2). From these facts, it can be seen that temporal filtering that performs correction using signal data acquired in a plurality of frames for each pixel of a plurality of pixels is more suitable for removing the bright spot than the correction process based on spatial comparison between different pixels of an image acquired from the signal data acquired in one frame. Given the foregoing, a correction process for dotted noise (bright spots) occurring at random both temporally and spatially in the radiation imaging apparatus 100 will be described below.

First, imaging is performed while uniformly irradiating the radiation without placing the object, and signal data 104d1 to 104dn, which are output from the ADC 102 and are to be gain map original images, are stored in the memory 104. As illustrated in FIG. 3, the signal data 104d1 to 104dn to be the gain map original image are acquired across a plurality of frames. Subsequently, the acquired signal data 104d1 to 104dn are read generally in parallel, on which the corrector 301 performs temporal filtering 106. The temporal filtering 106 by the corrector 301 performs a Hampel filter algorithm (Hampel filtering) described below and compares signal values at different time points of pixels placed at the same positions, and excludes outstanding values. Subsequently, the data with outstanding signal values excluded therefrom are averaged and stored in the memory 104 as the gain map 104g. According to the series of processing, bright spots included in the signal data 104d1 to 104dn are suppressed, thereby reducing the effect on the gain map 104g. In this case, there is a possibility that the effect of the bright spots included in the signal data 104d1 to 104dn on the gain map 104g is completely removed. In addition, averaging the plurality of signal data 104d1 to 104dn allows for mitigation of the effect of random noise included in the respective signal data 104d1 to 104dn, whereby the gain map 104g, which is closer to the true sensitivity distribution gain, can be acquired.

Here, a memory bandwidth of the radiation imaging apparatus 100 required for gain calibration operation will be described. Storing of the signal data 104d1 to 104dn, which are gain map original images, in the memory 104 must be performed with an approximately same speed as the speed of reading the signal data from the ADC 102, in order to maintain the system operation speed of the radiation imaging system SYS (radiation imaging apparatus 100). Although there is a difference in the operation direction between writing the signal data to the memory 104 and reading the gain map 104g from the memory 104, the amount of data being handled is equivalent to those in the bandwidth required for the aforementioned gain correction process in that the data covers only a single system of an image.

Next, the data being handled when performing the temporal filtering 106 is a sum of reading all the gain map original images (signal data 104d1 to 104dn, described here as there are five systems of signal data 104d1 to 104d5), and writing of the gain map 104g4, which adds up to six systems. However, since there is usually no demand for observing the temporal filtering 106 in real time, the process is not required to be performed in real time, and there is almost no harm in performing the process slowly within an available range of memory bandwidths. The same goes for an increased number of gain map original images. Based on the foregoing description, it is possible to acquire the gain map 104g, which is close to the true sensitivity distribution gain, without increasing the bandwidth of the memory 104.

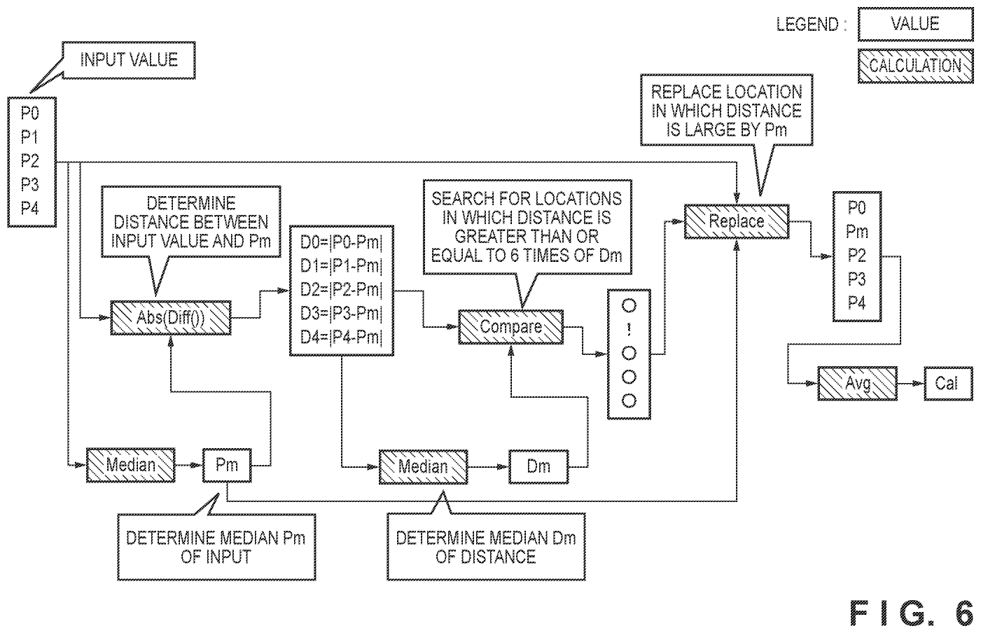

Subsequently, the temporal filtering 106 using a Hampel filter algorithm will be described, referring to FIG. 6. FIG. 6 illustrates a process performed in the temporal filtering 106 as a block diagram. As has been described above, all the gain map original images having been read, and the input unit of the processing receives input of signal values P0 to P4 acquired at different time points of pixels located at same positions in a plurality of frames. First, a process for determining a median Pm of all the signal values P0 to P4 is performed. The median Pm, which is a value representative of all the signal values P0 to P4 and inserted in place of the excluded outstanding signal values in the subsequent processing, is required to be an indicator less susceptible to the outstanding signal values. Therefore, the Hampel filter algorithm employs the median Pm instead of the mean value. Although the arithmetic operation of the median Pm, which involves a sorting process, requires a long time for calculation and may become a bottleneck in the execution speed of the temporal filtering 106, there is little demand for real time as described above, and therefore the calculation time is less likely to be problematic.

Following the calculation of the median Pm, distances (difference) D0 to D4 between all the signal values P0 to P4 and Pm is acquired. When there are no bright spots included therein, which are dotted noise, in the signal values P0 to P4, respective distances D0 to D4 are values close to each other. When a signal value due to a bright spot is included in the signal values P0 to P4, only the distance D of that signal value (e.g., any of D0 to D4) indicates an outstanding value. As a reference for determination thereof, a distance data median Dm is determined as a piece of data representative of the distances D0 to D4. Here, the distance data median Dm, which is the median, is used instead of the mean, in order to be less susceptible to outstanding distance data.

Next, the outstanding signal value is detected and replaced. The detection criteria for the outstanding point is on the basis that the distance D0 to D4 is larger than a value of Dm×determination reference coefficient. The determination reference coefficient may be preliminarily defined considering the random noise characteristics or the like of the sensor array 101. In the example illustrated in FIG. 6, a determination reference coefficient of 6 is employed. The signal value of the signal data of the pixel determined to be an outstanding signal value is excluded and the signal value is replaced by the median Pm. In the example illustrated in FIG. 6, the signal value P1 is replaced by the median Pm. The aforementioned process is the Hampel filter algorithm. In order to acquire the gain map 104g, outputs of the signal values acquired by the Hampel filter algorithm are averaged. Furthermore, the signal values of the pixels at reference positions of the gain is normalized to become 1.0 fold, whereby respective signal values of the gain map 104g are acquired.

Although the temporal filtering 106 in the corrector 301 is used to generate the gain map 104g, it is not used when imaging the object. This is mainly because whereas the sensitivity distribution of the sensor array 101 is less likely to temporarily change, the shape of the object may temporarily change. When an object image accompanied with motion is passed through the Hampel filter, the moving part is determined to be data indicating an outstanding signal value and excluded, and may be replaced by past data, resulting in occurrence of an after image. Therefore, the Hampel filter is not used when observing the object, and another correction process performs suppression of dotted noise such as bright spots that occur when imaging the object. In the correction process performed by the corrector 301 illustrated in FIG. 3, spatial filtering 109 is used for this purpose. The spatial filtering 109, which is a process for removing dotted noise such as bright spots by detecting spatial features in a single image included in the signal data acquired in one frame, has a better low-latency than the temporal filtering 106 and thus is suitable for real-time observation of images. The spatial filtering 109 may be implemented by the corrector 301 in the IC 103 as illustrated in FIG. 3, or may be performed on image data output from the radiation imaging apparatus 100 such as the image display apparatus 110. Additionally, for example, in a case where remainder of sudden bright spots are tolerable in the observation, the spatial filtering 109 may be turned off, or implementation of the spatial filtering 109 may be omitted.

In the foregoing manner, the corrector 301 performs a correction process for acquiring the gain map 104g for gain correction used in the gain correction process 108 without placing the object, and a correction process including gain correction (gain correction process 108) using the gain map 104g. On this occasion, correction processes performed on dotted noise such as bright spots that occur at random both temporally and spatially are different for the correction process for acquiring the gain map 104g and the correction process including the gain correction process 108, for example, for when imaging the object. In the correction process for acquiring the gain map 104g on this occasion, the corrector 301 uses the signal data acquired in the plurality of frames. Additionally, in the correction process including the gain correction process 108, the corrector 301 uses the signal data acquired in at least one frame. For example, in a correction process for video image capturing, a correction process including the gain correction process 108 and the spatial filtering 109 may be performed using signal data acquired in one frame. As a result, the number of frames for acquiring signal data to be used in the correction process for acquiring the gain map 104g may exceed the number of frames for acquiring signal data to be used in the correction process including the gain correction process 108. Therefore, the processing speed of the correction process for acquiring the gain map 104g may become slower than the processing speed of the correction process including the gain correction process 108, for example, for when capturing video image. However, the process for acquiring the gain map 104g is performed during a calibration period when starting up the radiation imaging apparatus 100, for example, and therefore is less likely to affect the user experience of the operator.

As has been described above, the directional filtering in the present embodiment suppresses, by the temporal filtering 106, the effect of bright spots occurred by radiation directly incident on the sensor array without being converted into light by the scintillator 105, and acquires the gain map 104g. Therefore, it is possible to prevent, in the gain correction process 108, occurrence of constant dark spots in the acquired radiation image. In addition, when imaging the object, radiation images are provided by a different correction process path from the correction process for acquiring the gain map 104g, whereby object observation with less display delay can be realized. In addition, it is not necessary to enhance the bandwidth of the memory 104 of the radiation imaging apparatus 100, making the radiation imaging apparatus 100 inexpensively available.

In the process illustrated in FIG. 3, the spatial filtering 109 is not performed in the correction process for acquiring the gain map 104g. However, the foregoing is not limiting and the spatial filtering 109 may be performed after having performed the temporal filtering 106 when acquiring the gain map 104g. The process of averaging the outputs of the signal values acquired by the temporal filtering 106 may be performed either before or after the spatial filtering 109.

In addition, although the corrector 301 is illustrated in FIG. 3 as being provided in the IC 103, this is not limiting. For example, at least a part of the correction process performed by the corrector 301 may be implemented on another integrated circuit such as an ASIC or an FPGA provided separately from the IC 103. In addition, the corrector 301 may be provided independently of the IC 103. The same goes for the following description.

Here, the memory bandwidth required by the memory 104 in the temporal filtering 106 will be described again. As illustrated in FIG. 6, in Hampel filtering, input values (signal values P0 to P4) are referenced at a plurality of steps of the process. Specifically, the input values are referenced at respective steps of calculating the median Pm, calculating the distance D (distances D0 to D4), and determining the population of averaging. Configuring the corrector 301 to acquire input values from the memory 104 at each of these steps causes a three-fold increase of components related to reading of the gain map original image (signal data 104d1 to 104dn), among the aforementioned discussion of memory bandwidths. In other words, the amount of data to be handled turns out to correspond not to 5+1=6 systems but to 5×3+1=16 systems.

As has been described above, the process need not be performed in real time, and therefore the increase in the number of systems of data does not immediately wreck the operation of the radiation imaging apparatus 100 as a system. However, when the number of gain map original images increases, there may arise a possibility of affecting user experience of the operator. For example, when the number of gain map original images increases from the aforementioned 5 to 30, the amount of data to be handled increases by the difference between 30+1=31 systems and 30×3+1=91 systems, requiring an extra amount of time as much as the difference to perform the temporal filtering 106. This may result in a standby period during which the operator cannot use the radiation imaging apparatus 100, which may need to be suppressed.

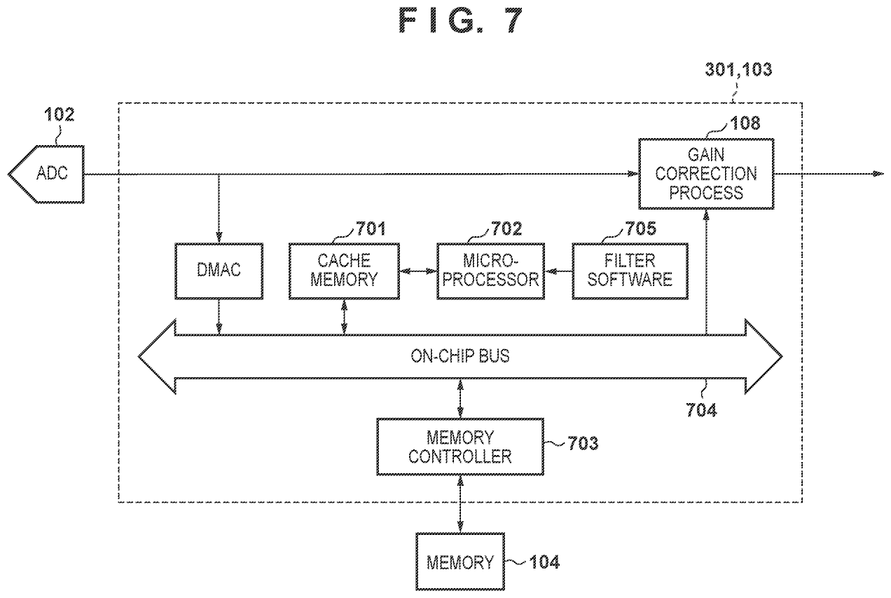

In view of the foregoing, the corrector 301 of the IC 103 in the present embodiment is configured as illustrated in FIG. 7, in order to perform the temporal filtering 106. The corrector 301 of the radiation imaging apparatus 100 according to the present embodiment includes therein an on-chip bus 704 and a memory controller 703, arbitrates memory access requests from respective blocks in the IC 103, and reads from or writes into the memory 104. The IC 103 also includes a microprocessor 702 and performs an algorithm of the temporal filtering 106 illustrated as filter software 705. Performing the algorithm of the temporal filtering 106 causes the microprocessor 702 to issue a read request of gain map original images (signal data 104d1 to 104dn), and at the middle of the path through which the request is processed, a cache memory 701 is provided. The cache memory 701 temporarily holds the content of the gain map original images read from the memory 104. When read requests of a same address are repeatedly issued from the microprocessor 702, the temporarily stored data is returned to the microprocessor 702 without the data being read from the memory 104.

Subsequently, there will be described the operation of the microprocessor 702 and the cache memory 701 when performing the filter algorithm of the temporal filtering 106. The microprocessor 702, when performing the step of calculating the median Pm, issues a read request to read the gain map original images (signal data 104d1 to 104dn). When the data stored in a corresponding memory address is not held in the cache memory 701, the read request passes through the cache memory 701 and reaches the memory controller 703 via the on-chip bus 704. The memory controller 703 reads the data from the specified address of the memory 104 and returns the read-out data. The read-out data travels in the reverse direction along the path through which the read request has passed, and reaches the microprocessor 702. On this occasion, when the data read from the memory 104 passes through the cache memory 701, a combination of the reading address and the data is temporarily stored in the cache memory 701. The microprocessor 702 calculates the median Pm using the data of the received gain map original images, and subsequently performs a step of calculating the distance D (distances D0 to D4). In the step of calculating the distance D, the microprocessor 702 issues a read request for the same address as the step of calculating the median Pm again. The read request is not transferred to the memory controller 703 because corresponding data is held in the cache memory 701, and the cache memory 701 returns the corresponding data to the microprocessor 702. The microprocessor 702 uses the received data to calculate the distance D. Hereinafter, determination of the population of averaging is also performed in a similar manner, using the data returned from the cache memory 701. In the aforementioned series of operation, the cache memory 701 returns data in a shorter time than reading data from the memory 104, which allows for reducing the processing time in comparison with the configuration that reads data from the memory 104 each time.

Furthermore, the cache memory 701 may collectively manage signal values of successive, for example 16 pixels, instead of separately managing respective signal values of pixels arranged on the sensor array 101. The collection of signal values is referred to as a cache line. The cache memory 701 transfers, to the memory 104, a read request for the data of successive 16 pixels arranged in a manner filling a single cache line, at the time point when data of the signal value of one pixel is requested. Accordingly, the read request to the memory 104 turns out be a burst access, reducing the overhead of communication between the memory controller 703 and the memory 104. Also in such an aspect, the cache memory 701 contributes to increasing the speed of the temporal filtering 106 of the radiation imaging apparatus 100.

The cache memory 701 has a finite capacity and eventually reaches a time point of running out its capacity as the temporal filtering 106 proceeds while changing the position of the pixel of interest. On this occasion, the cache memory 701 secures available capacity by erasing the cache memory data in chronological order. Let us consider a case where the number of cache lines is smaller than the number of gain map original images, for example a case where there are four cache lines when using, for example, five gain map original images (five pieces of signal data) as described above. In such a case, the cache line including the signal value P0 is deleted at the time point when the microprocessor 702 requests the signal value P4, whereby it becomes necessary to acquire the signal value P0 from the memory 104 again, depriving the benefit of temporarily storing data (signal values) in the cache memory 701. In order to avoid this situation, the number of cache lines included in the cache memory 701 may be larger than the number of gain map original images. Accordingly, the effect of providing the cache memory 701 is enhanced.

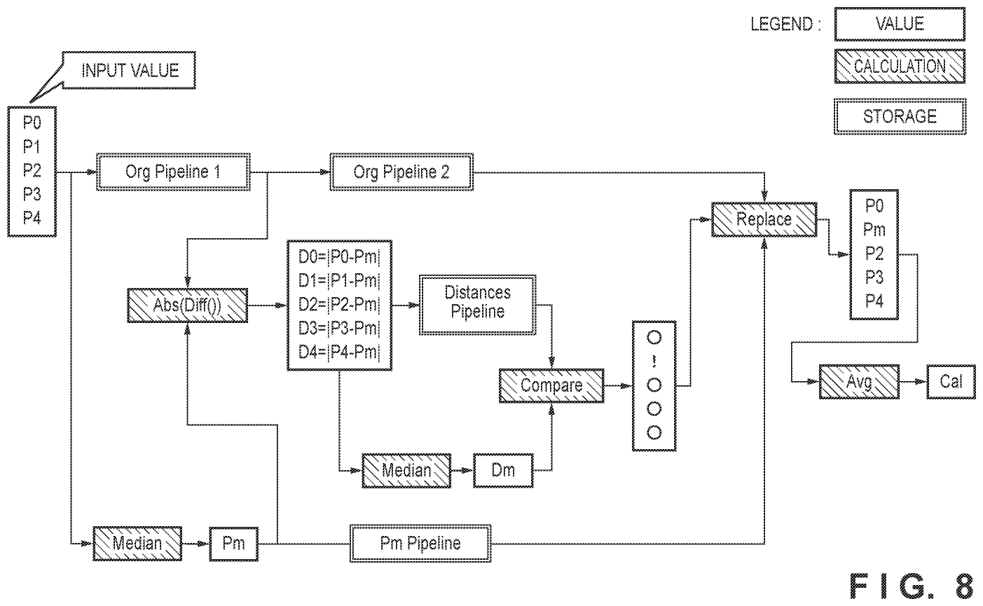

Another configuration distinct from the configuration of providing the cache memory 701 with regard to the problem of increasing memory bandwidth will be further described, referring to FIG. 8. FIG. 8 illustrates an implementation of the process illustrated in FIG. 6 as a computation pipeline of hardware. Compared with the process illustrated in FIG. 6, a “storage” component is added. The component has a function of storing (temporarily storing) input values or calculation values when they need to be referenced in a plurality of steps of calculation, until a timing of being actually referenced. This function may be configured using an FIFO memory as an entity. The Org Pipeline 1 illustrated in FIG. 8 is used for temporarily storing the input signal values P0 to P4 until the step of calculating the median Pm ends and calculation of the distance D (distances D0 to D4) starts. Similarly, the Org Pipeline 2 temporarily stores the input signal values P0 to P4 until the step of determining the population of averaging. Equipping these elements reduces the necessity of accessing the memory 104 and reading the signal data (signal values) again.

Since the process illustrated in FIG. 8 is configured with a pipeline, it is possible to start processing of the next pixel before the result of processing a certain pixel by the temporal filtering 106 reaches an exit Cal of the pipeline. For example, it becomes possible to start the step of calculating the median Pm of the next pixel data simultaneously at the time point when calculation of the median Pm of the head pixel is completed and subsequently entering the step of calculating the distance D together with the output of the Org Pipeline 1. The configuration allows for calculation by the temporal filtering 106 using the memory bandwidth to maximum extent without increasing the load on the memory 104.

Having provided the foregoing description of the embodiments of the present disclosure, it goes without saying that the present embodiment is not limited to such embodiments, and the aforementioned embodiments can be modified or combined as appropriate to an extent not departing from the gist of the present invention. For example, all the processes in the present embodiments are implemented on the IC 103. The IC 103 may be implemented by a programmable circuit such as an FPGA, or may be realized as an ASIC which is a fixed circuit, or may be realized by a combination of a microprocessor which is a fixed circuit and software. In addition, although the foregoing description is based on using the Hampel filter algorithm in the temporal filtering 106, the present invention is not limited thereto. Any filtering process that temporally refers to data for a plurality of times and suppresses the effect of outstanding signal values (so-called outliers) can exert the effect of the present disclosure. For example, a method such as the 3σ method may be used for detecting outstanding signal values.

In addition, although the description using FIG. 7 referred to a software-based example and the description using FIG. 8 referred to a hardware-based example, respectively, with respect to implementation of the temporal filtering 106, it is also possible to provide an intermediate hybrid configuration. For example, calculation for acquiring the median Pm is a heavy-load process including a sorting process, and therefore a configuration is conceivable which combines the microprocessor and the sort-supporting hardware. In addition, for example, the cache memory 701 can be realized by controlling the on-chip scratch pad memory by the software. In addition, the configuration of the process illustrated in FIG. 8 may be configured by a different type of pipeline in order to limit the circuit scale. For example, there may be a configuration in which blocks that derive two medians, namely the median Pm and the distance data median Dm, are united into a single block so that calculations of the median Pm and the distance data median Dm are performed alternately. However, in this case, the efficiency of the entire pipeline may decrease by 50%. It is suggested that a suitable configuration should be used as appropriate in accordance with the requirement to processing speed or circuit scale of the radiation imaging apparatus 100.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-034665, filed Mar. 4, 2021 which is hereby incorporated by reference herein in its entirety.

What is claimed is:

1. A radiation imaging apparatus comprising:
a scintillator;
a plurality of pixels configured to respectively detect light converted by the scintillator from radiation; and
a corrector configured to perform a correction process on signal data based on signals output from the plurality of pixels, wherein
the corrector is configured to perform a first correction process for acquiring a gain map for gain correction without placing an object, and a second correction process including gain correction using the gain map, and
correction processes performed on dotted noise that occur at random both temporally and spatially are different for the first correction process and the second correction process.

2. The radiation imaging apparatus according to claim 1, wherein
the corrector is configured to:
perform, in the first correction process, a correction process using the signal data acquired in a plurality of frames; and
perform, in the second correction process, a correction process using the signal data acquired in at least one frame, and
number of frames for acquiring the signal data to be used in the first correction process is larger than number of frames for acquiring the signal data to be used in the second correction process.

3. The radiation imaging apparatus according to claim 1, wherein, in the first correction process, the corrector is configured to perform temporal filtering that performs correction using the signal data acquired in a plurality of frames for each of the plurality of pixels.

4. The radiation imaging apparatus according to claim 3, wherein the corrector is configured to not perform the temporal filtering in the second correction process.

5. The radiation imaging apparatus according to claim 3, wherein the temporal filtering detects, among the signal data acquired in a plurality of frames for each of the plurality of pixels, the signal data of a frame that outputs an outstanding signal value, and performs a correction process on the signal value of the signal data of the detected frame.

6. The radiation imaging apparatus according to claim 5, wherein the temporal filtering replaces the signal value of the signal data of the detected frame by a median value of the signal values of the signal data acquired in a plurality of frames for each of the plurality of pixels.

7. The radiation imaging apparatus according to claim 3, further comprising a memory configured to store the signal data acquired for each frame, wherein
the corrector is configured to perform, in the temporal filtering, a correction process referring to the plurality of pieces of signal data stored in the memory.

8. The radiation imaging apparatus according to claim 7, wherein the corrector further includes a cache memory configured to temporarily store the signal data that has been read out from the memory, and is configured to perform, in the temporal filtering, a correction process referring to the plurality of pieces of signal data stored in the cache memory.

9. The radiation imaging apparatus according to claim 1, wherein the corrector is configured to perform, in the second correction process, spatial filtering that performs spatial correction using the signal data acquired in one frame.

10. The radiation imaging apparatus according to claim 9, wherein the corrector is configured to not perform the spatial filtering in the first correction process.

11. The radiation imaging apparatus according to claim 3, wherein
the corrector is configured to:
perform, in the second correction process, spatial filtering that performs spatial correction using the signal data acquired in one frame; and
perform, in the first correction process, the spatial filtering after having performed the temporal filtering.

12. The radiation imaging apparatus according to claim 1, wherein processing speed of the first correction process is lower than processing speed of the second correction process.

13. The radiation imaging apparatus according to claim 1, wherein the second correction process is a correction process to be performed when a video image is captured.

14. A radiation imaging system comprising:
the radiation imaging apparatus according to claim 1; and
a radiation source configured to irradiate radiation on the radiation imaging apparatus.
